(19) United States
(12) Patent Application Publication
(10) Pub. No.: US 2009/0307681 A1
(43) Pub. Date: Dec. 10, 2009

(54) Wireless Network and Methods of Wireless Communication For Ophthalmic Surgical Consoles

(76) Inventors: Ryan Armado, Chino Hills, CA (US); Kevin King, Santa Ana, CA (US); Kurt Leukanech, Laguna Niguel, CA (US); Mark Young, Laguna Niguel, CA (US)

Correspondence Address: ALCON, IP LEGAL, TB4-8, 6201 SOUTH FREEWAY, FORT WORTH, TX, 76134, US

(21) Appl. No.: 12/478,000

(22) Filed: Jun. 4, 2009

Related U.S. Application Data
(60) Provisional application No. 61/059,074, filed on Jun. 5, 2008.

(51) Int. Cl. G06F 9/44; A61B 17/00; G06F 15/16; G06F 9/00

(52) U.S. Cl. 717/171; 606/1; 709/227; 709/219; 726/11

(57) ABSTRACT

A wireless network of surgical consoles and other equipment is used to transfer data. A surgical console has a system processor running software that operates the surgical console and a wireless processor coupled to the system processor. The wireless processor runs software that facilitates wireless communication. A transceiver is coupled to the wireless processor. The system processor is physically separate from the wireless processor. The wireless processor is used to transfer data to other devices and receive software updates.

### RELATED APPLICATIONS

This Application claims priority to U.S. Provisional Patent Application No. 61/059,074 filed on Jun. 5, 2008.

### BACKGROUND OF THE INVENTION

The present invention relates to ophthalmic surgical consoles and more particularly to systems and methods for wirelessly connecting surgical consoles to each other and to other equipment.

Anatomically, the eye is divided into two distinct parts—the anterior segment and the posterior segment. The anterior segment includes the lens and extends from the outermost layer of the cornea (the corneal endothelium) to the posterior of the lens capsule. The posterior segment includes the portion of the eye behind the lens capsule. The posterior segment extends from the anterior hyaloid face to the retina, with which the posterior hyaloid face of the vitreous body is in direct contact. The posterior segment is much larger than the anterior segment.

The posterior segment includes the vitreous body—a clear, colorless, gel-like substance. It makes up approximately two-thirds of the eye's volume, giving it form and shape before birth. It is composed of 1% collagen and sodium hyaluronate and 99% water. The anterior boundary of the vitreous body is the anterior hyaloid face, which touches the posterior capsule of the lens, while the posterior hyaloid face forms its posterior boundary, and is in contact with the retina. The vitreous body is not free-flowing like the aqueous humor and has normal anatomic attachment sites. One of these sites is the vitreous base, which is a 3-4 mm wide band that overlies the ora serrata. The optic nerve head, macula lutea, and vascular arcade are also sites of attachment. The vitreous body's major functions are to hold the retina in place, maintain the integrity and shape of the globe, absorb shock due to movement, and to give support for the lens posteriorly. In contrast to aqueous humor, the vitreous body is not continuously replaced. The vitreous body becomes more fluid with age in a process known as syneresis. Syneresis results in shrinkage of the vitreous body, which can exert pressure or traction on its normal attachment sites. If enough traction is applied, the vitreous body may pull itself from its retinal attachment and create a retinal tear or hole.

Various surgical procedures, called vitreo-retinal procedures, are commonly performed in the posterior segment of the eye. Vitreo-retinal procedures are appropriate to treat many serious conditions of the posterior segment. Vitreo-retinal procedures treat conditions such as age-related macular degeneration (AMD), diabetic retinopathy and diabetic vitreous hemorrhage, macular hole, retinal detachment, epiretinal membrane, CMV retinitis, and many other ophthalmic conditions.

A surgeon performs vitreo-retinal procedures with a microscope and special lenses designed to provide a clear image of the posterior segment. Several tiny incisions just a millimeter or so in length are made on the sclera at the pars plana. The surgeon inserts microsurgical instruments through the incisions such as a fiber optic light source to illuminate inside the eye, an infusion line to maintain the eye's shape during surgery, and instruments to cut and remove the vitreous body.

Another common surgical procedure, cataract removal and lens replacement, is performed on the anterior segment of the eye. The eye's natural lens is composed of an outer lens capsule enclosing a lens cortex. Since the human eye functions to provide vision by transmitting light through a clear outer portion called the cornea, and focusing the image by way of a clear crystalline lens onto a retina, the quality of the focused image depends on many factors including the transparency of the lens. When age or disease causes the lens to become less transparent, vision deteriorates because of the diminished light which can be transmitted to the retina. This deficiency in the lens of the eye is medically known as a cataract. An accepted treatment for this condition is cataract surgery which involves the removal and replacement of the lens cortex by an artificial intraocular lens (IOL).

In the United States, the majority of cataractous lenses are removed by a surgical technique called phacoemulsification. During this procedure, an incision of a few millimeters in size is made in the cornea or sclera. By way of the incision, a thin phacoemulsification cutting tip is inserted into the diseased lens and vibrated ultrasonically. The vibrating cutting tip liquefies or emulsifies the lens cortex material so that it may be aspirated out of the eye. The diseased lens material, once removed, is replaced by an IOL.

The IOL is injected into the eye through the same small incision used to remove the diseased lens cellular material. The IOL is placed in an IOL injector in a folded state to avoid enlarging the incision. The tip of the IOL injector is inserted into the incision, and the lens is delivered into the lens capsular bag.

Sophisticated surgical machines are used to perform these various anterior and posterior segment procedures. Such machines have computer controlled consoles to which surgical instruments are attached. Often, a hospital or surgical suite has more than one of these machines. Typically, these machines are stand-alone units and are not networked together—even though each machine includes computer equipment. With the growing popularity of wireless communication devices, networking these machines to each other and to other computers may provide benefits. It would be desirable to have a wireless network that includes these surgical machines to facilitate communication between them and other computers.

### SUMMARY OF THE INVENTION

In one embodiment consistent with the principles of the present invention, the present invention is a surgical console with a system processor running software that operates the surgical console and a wireless processor coupled to the system processor. The wireless processor runs software that facilitates wireless communication. A transceiver is coupled to the wireless processor. The system processor is physically separate from the wireless processor.

In another embodiment consistent with the principles of the present invention, the present invention is a network that includes first and second surgical consoles. The first surgical console has a first system processor coupled to a first wireless processor. The first wireless processor is coupled to a first transceiver. The second surgical console has a second system processor coupled to a second wireless processor. The second wireless processor is coupled to a second transceiver. A wireless connection can be established between the first surgical console and the second surgical console.

In another embodiment consistent with the principles of the present invention, the present invention is a method of wirelessly transferring data between surgical consoles comprising reading user preferences from memory in a first surgical console; transferring the user preferences to a first wireless processor coupled to a first system processor in the first surgical console; and transmitting the user preferences from a first transceiver coupled to the first wireless processor.

In another embodiment consistent with the principles of the present invention, the present invention is a method of updating software running on a surgical console, the method comprising: receiving, by way of a wireless processor, a software update that has been wirelessly transmitted; transferring the software update from the wireless processor to a system processor located in a surgical console; and updating the software running on the system processor with the software update.

In another embodiment consistent with the principles of the present invention, the present invention is a method of wirelessly transferring data from a surgical console, the method comprising: storing operational data in memory coupled to a system processor located in a surgical console; reading the operational data from the memory; sending the operational data to a wireless processor coupled to the system processor; and wirelessly transmitting the operational data.

It is to be understood that both the foregoing general description and the following detailed description are exemplary and explanatory only and are intended to provide further explanation of the invention as claimed. The following description, as well as the practice of the invention, set forth and suggest additional advantages and purposes of the invention.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 is a diagram of a wireless network including surgical consoles and other equipment according to the principles of the present invention.

FIG. 2 is a block diagram of a processor architecture for a surgical machine according to the principles of the present invention.

FIG. 3 is a block diagram of a method of maintaining user preferences wirelessly among consoles according to the principles of the present invention.

FIG. 4 is a block diagram of a method of providing software updates to consoles wirelessly according to the principles of the present invention.

FIG. 5 is a block diagram of a method of collecting service data wirelessly according to the principles of the present invention.

### DETAILED DESCRIPTION OF THE PREFERRED EMBODIMENTS

Reference is now made in detail to the exemplary embodiments of the invention, examples of which are illustrated in the accompanying drawings. Wherever possible, the same reference numbers are used throughout the drawings to refer to the same or like parts.

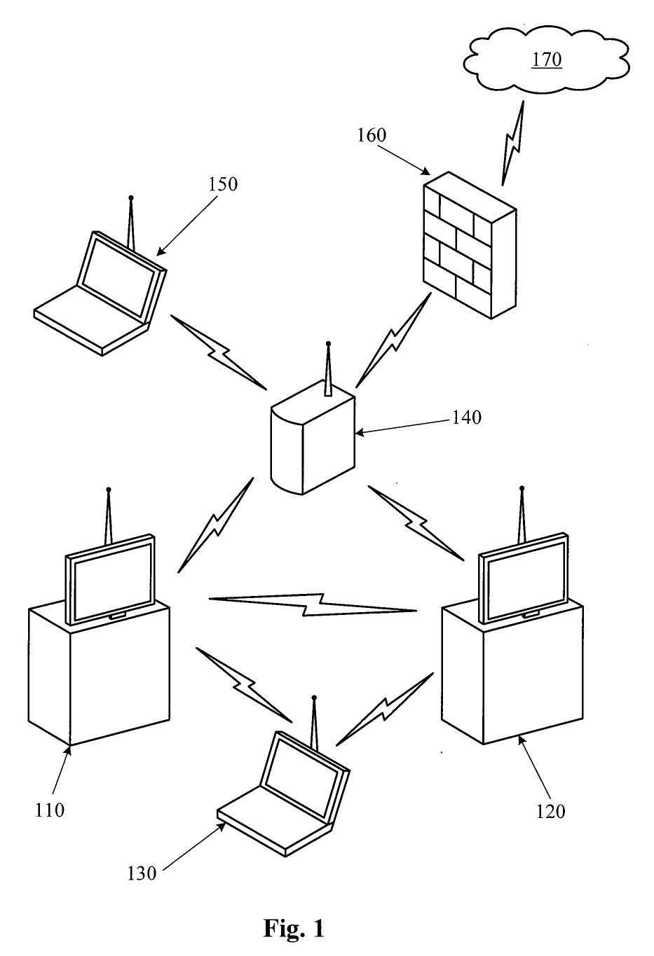

FIG. 1 is a diagram of a wireless network including surgical consoles and other equipment according to the principles of the present invention. In this example, two surgical consoles 110 and 120 and a server 140 are located in a hospital environment. Surgical consoles 110 and 120 are typically located in different operating rooms. Server 140 is typically located in the same building as surgical consoles 110 and 120. For example, a facility that performs a large number of cataract operations in a day has two or more cataract surgical consoles (like consoles 110 and 120) located in adjacent operating rooms. Since cataract procedures are generally performed in a matter of fifteen minutes or so, several patients are typically prepped for the procedure in separate operating rooms, and the ophthalmic surgeon goes from one room to another to perform the procedure on the different patients. For vitreoretinal surgery (which is much more time consuming and complicated), an eye clinic may have several vitreoretinal surgical consoles (such as consoles 110 and 120) located in different operating rooms. In such a case, different surgeons may use different consoles in different operating rooms simultaneously.

In FIG. 1, surgical console 110 is wirelessly coupled to surgical console 120, laptop computer 130 and server 140. Surgical console 120 is wirelessly coupled to surgical console 110, laptop computer 130 and server 140. Laptop computer 130 is wirelessly coupled to surgical console 110 and surgical console 120. Server 140 is wirelessly coupled to surgical console 110, surgical console 120, laptop computer 150, and the internet 170 (through firewall 160). Laptop computer 150 is wirelessly coupled to server 140. Firewall 160 is configured to protect the components that reside at the hospital or surgical suite (surgical consoles 110 and 120 and server 140) and the laptop computers 130 and 150 to which they connect.

While only two consoles 110 and 120 and one server 140 are shown, any number of consoles, servers, and peripheral equipment may be present. Examples of peripheral equipment that may be connected to a console include, but are not limited to: printers, monitors, cameras, bar code readers, microscopes, and the like. In addition, server 140 (and other servers, if present) may be connected to a hospital network, a multi-site network, or the internet.

Any of a number of different wireless protocols can be used to wirelessly connect the components depicted in FIG. 1. Examples of such protocols include, but are not limited to: 802.11g, WiMAX, Wi-Fi, firewire, cellular, or any other wireless protocol. Further, more than one wireless protocol can be used. For example, console 110 may be wirelessly coupled to console 120 via an 802.11g protocol. Laptop computer 130 may be wirelessly coupled to consoles 110 and 120 via a firewire protocol. In this manner, one or more of the components depicted in FIG. 1 may have more than one wireless protocol enabled.

Laptop computers 130 and 150 may be any of a number of different laptop computers with a wireless feature. In other embodiments, laptop computers 130 and 150 may be implemented with any wireless device such as, for example, a handheld computer or wireless device, a cell phone device, a portable electronic device with a wireless feature, or the like.

Server 140 is typically a server computer such as those commonly found in computer networks. In this manner, server 140 is typically an intermediary computer whose primary functions are to communicate with other computing devices and store data. Server 140 includes a transceiver or other means of wirelessly connecting to other devices. As such, server 140 is capable of being part of a wireless network. However, in other embodiments of the present invention, server 140 may be implemented with any type of computer device.

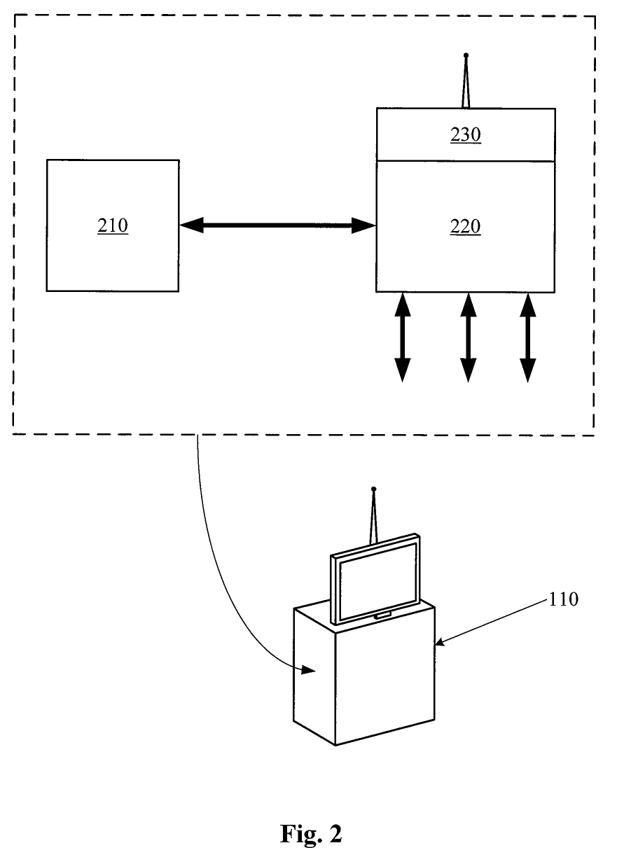

FIG. 2 a block diagram of a processor architecture for a surgical console according to the principles of the present invention. In FIG. 2, system processor 210 is connected to wireless processor 220. Wireless processor 220 is connected to transceiver 230. As such, console 110 (and console 120 as well) has a processor architecture in which the system processor 210 and the wireless processor 220 are separate processors.

System processor 210 may be any of a number of different types of microprocessors or microcontrollers. For example, system processor 210 may be a standard microprocessor such as those manufactured by Intel (e.g. Pentium) or AMD. System processor 210 may also be a multicore processor or other type of computing circuit. Wireless processor 220 supports a wireless protocol (such as, for example, 802.11g, 802.1X, cellular, etc.).

This two processor design (separate system processor and wireless processor) facilitates isolation of software development. Software for the console can be developed for system processor 210, and communications software can be developed for wireless processor 220. The use of two separate processors also increases the modularity of the components (in addition to the modularity of the software). As such, software on the two processors can be updated separately. Having wireless processor 220 separate from system processor 210 also provides complete isolation of the host file structure for security and safety purposes. Separate wireless processor 220 can function as a firewall protecting system processor 210. In addition, wireless processor 220 (and transceiver 230) may be embodied in a separate unit that can be connected to and disconnected from the console 110 (and system processor 210). In this manner, a separate box containing wireless processor 220 and transceiver 230 can be plugged into a connector that resides on console 110 and interfaces with system processor 210.

In another embodiment of the present invention, a single processor architecture may be used. In this embodiment, system processor 210 performs all of the wireless functions, and wireless processor 220 is not present. In yet another embodiment of the present invention, system processor 210 and wireless processor 220 are located on a single substrate or in a single IC package.

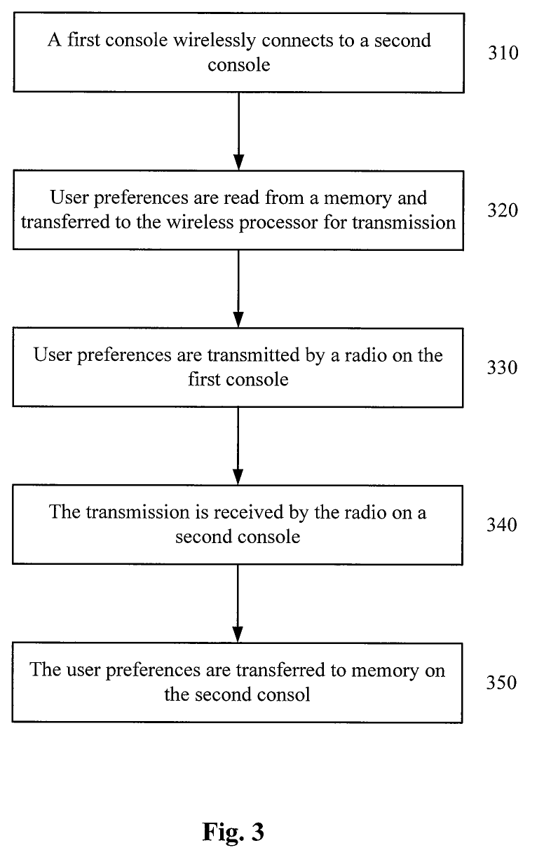

FIG. 3 is a block diagram of a method of maintaining user preferences wirelessly among consoles according to the principles of the present invention. In this method, the wireless architecture of FIG. 1 is leveraged to allow easy transfer of user preferences from one console 110 to another console 120. Surgical consoles, such as those used for eye surgery, are computerized machines that can be customized to meet a surgeon's particular needs. These user preferences vary from surgeon to surgeon. In an eye care facility that has more than one surgeon or for a surgeon that uses more than one machine, it is desirable to match a surgeon's particular preferences with the machine the surgeon is using. Wirelessly transferring user preferences among or between consoles provides this benefit.

User preferences may be transferred directly between consoles (110 and 120), from one console to another (110 and 120) via server 140, or from one console to another (110 and 120) via any intermediate device or devices (like laptop 130). In addition, user preferences may reside on intermediate devices (like server 140 or laptop 130) or on a device connected to the internet 170.

In 310, a first console wirelessly connects to a second console. This wireless connection can be direct or through an intermediate device or devices. In 320, user preferences are read from a memory and transferred to the wireless processor for transmission. In 330, the user preferences are transmitted by a transceiver on the first console. In 340, the transmission is received by the transceiver on a second console. In 350, the user preferences are transferred to memory on the second console where they are available for use by the system processor on the second console.

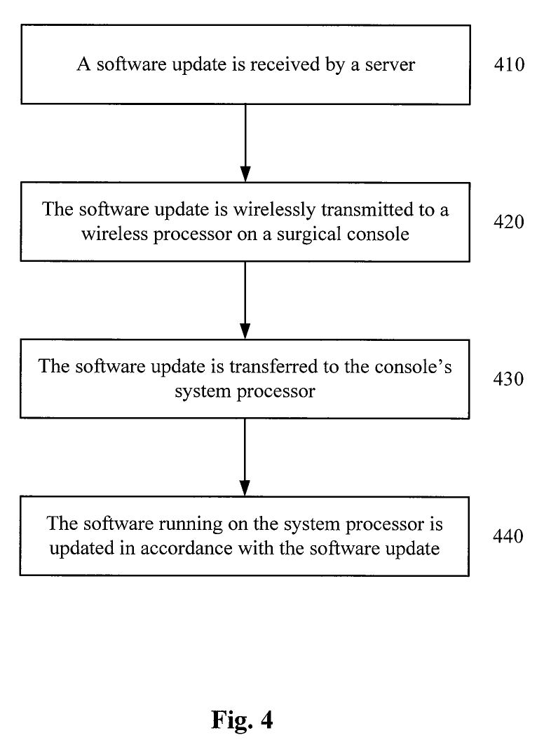

FIG. 4 is a block diagram of a method of providing software updates to consoles wirelessly according to the principles of the present invention. In this method, the wireless architecture of FIG. 1 is leveraged to allow software updates on surgical consoles. Traditionally, a service technician personally visits a surgical console to perform a software update. The technician inserts a compact disc with the software update into the console, and the update is copied to the console. Such a procedure is time consuming and expensive. Wirelessly transmitting software updates is much more efficient.

Software updates can be sent directly to one or more consoles from a service technician's laptop via a wireless connection. In this manner, a service technician may visit a hospital or surgical suite and wirelessly broadcast a software update to all consoles at that location or to a server at that location. Software updates may also be sent to a console via a server. Such software updates can be scheduled at any time. Further, software updates can originate from the console manufacturer, be transmitted over the internet to a server at a hospital or surgical suite, and then be sent from the server to the console. These software updates can be targeted at software running on the system processor, a sub-processor, or the wireless processor.

In 410, a software update is received by a server. This software update can be sent to the server over the internet from a console manufacturer or servicer, or it can be sent to the server from a technician's computer. In 420, the software update is wirelessly transmitted to a wireless processor on a surgical console. In 430, the software update is transferred to the console's system processor. In 440, the software running on the system processor is updated in accordance with the software update.

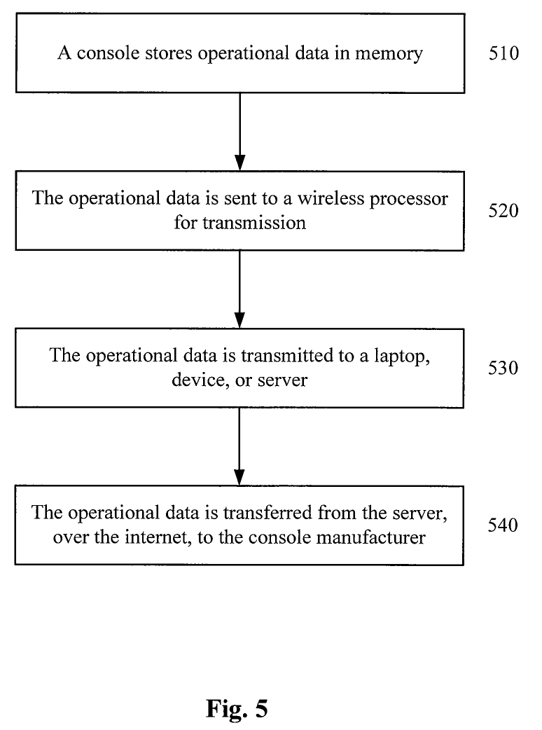

FIG. 5 is a block diagram of a method of collecting service data wirelessly according to the principles of the present invention. Data is often collected during a console's use. For example, the data about the various surgical parameters and the operation of the console is collected and stored in memory in the console. This provides data about how the console performs. Accordingly, this data can also be used to diagnose a console malfunction or problem. In addition, data about the operation of the console can be used to fine tune its operation or improve its performance. Such data is useful for a technician servicing the console.

This operational data can be transferred wirelessly directly from a console to a service technician's laptop. Alternatively, this data can be transferred wirelessly from a console to a server. From the server, it can be transferred to a laptop or other device, or it can be sent from the server, via the internet or other network, to a service technician or the console manufacturer.

In 510, a console stores operational data in memory. In 520, the operational data is sent to a wireless processor for transmission. In 530, the operational data is transmitted to a laptop, device, or server. In 540, the operational data is transferred from the server, over the internet, to the console manufacturer.

From the above, it may be appreciated that the present invention provides a system and methods for wirelessly networking ophthalmic surgical machines. The present invention provides a processor architecture that facilitates wireless communication between and with surgical machines. Methods of sharing information among machines, providing system updates, and accessing data are also disclosed. The present invention is illustrated herein by example, and various modifications may be made by a person of ordinary skill in the art.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A surgical console comprising:
a system processor running software that operates the surgical console;
a wireless processor coupled to the system processor, the wireless processor running software that facilitates wireless communication; and
a transceiver coupled to the wireless processor;
wherein the system processor is physically separate from the wireless processor.

2. A network comprising:
a first surgical console, the first surgical console comprising a first system processor coupled to a first wireless processor, the first wireless processor coupled to a first transceiver;
a second surgical console, the second surgical console comprising a second system processor coupled to a second wireless processor, the second wireless processor coupled to a second transceiver;
wherein a wireless connection can be established between the first surgical console and the second surgical console.

3. The wireless network of claim 2 further comprising:
a server comprising a server transceiver, the server transceiver wirelessly coupled to the first transceiver and the second transceiver.

4. The wireless network of claim 3 further comprising:
a firewall located between the server and the internet.

5. The wireless network of claim 2 further comprising:
a laptop computer wirelessly connected to the first surgical console.

6. A method of wirelessly transferring data between surgical consoles, the method comprising:
reading user preferences from memory in a first surgical console;
transferring the user preferences to a first wireless processor coupled to a first system processor in the first surgical console; and
transmitting the user preferences from a first transceiver coupled to the first wireless processor.

7. The method of claim 6 further comprising:
receiving the user preferences in a second transceiver coupled to a second wireless processor, the second wireless processor coupled to a second system processor in a second surgical console;
transferring the user preferences from the second wireless processor to the second system processor; and
storing the user preferences in memory in the second surgical console.

8. The method of claim 6 further comprising:
receiving the user preferences in a server; and
storing the user preferences in server memory;

9. The method of claim 8 further comprising:
reading the user preferences from the server memory;
transmitting the user preferences to a second transceiver coupled to a second wireless processor, the second wireless processor coupled to a second system processor in a second surgical console;
transferring the user preferences from the second wireless processor to the second system processor; and
storing the user preferences in memory in the second surgical console.

10. A method of updating software running on a surgical console, the method comprising:
receiving, by way of a wireless processor, a software update that has been wirelessly transmitted;
transferring the software update from the wireless processor to a system processor located in a surgical console; and
updating the software running on the system processor with the software update.

11. The method of claim 10 further comprising:
wirelessly transmitting the software update from a server to the wireless processor.

12. The method of claim 11 further comprising:
transmitting the software update over the internet to the server.

13. The method of claim 10 further comprising:
wirelessly transmitting the software update from a laptop computer to the wireless processor.

14. A method of wirelessly transferring data from a surgical console, the method comprising:
storing operational data in memory coupled to a system processor located in a surgical console;
reading the operational data from the memory;
sending the operational data to a wireless processor coupled to the system processor; and
wirelessly transmitting the operational data.

15. The method of claim 14 further comprising:
receiving the operational data; and
using the operational data to service the surgical console.

16. The method of claim 15 wherein receiving the operational data further comprises:
receiving the operational data on a laptop computer.

17. The method of claim 15 wherein receiving the operational data further comprises:
receiving the operational data on a server.
